(19) United States
(12) Patent Application Publication
(10) Pub. No.: US 2011/0265792 A1
(43) Pub. Date: Nov. 3, 2011

(54) OXYGEN TUBING

(76) Inventors: GINA CRAWFORD, Dobbins, CA (US); Warren Crawford, Dobbins, CA (US)

(21) Appl. No.: 12/772,332

(22) Filed: May 3, 2010

(51) Int. Cl. A61M 16/08

(52) U.S. Cl. 128/204.18

(57) ABSTRACT

The present invention relates to oxygen tubing for an oxygen concentrator comprising: a spectrum of colors over the surface tubing, where the spectrum extends over the entire length of the tubing. The spectrum of colors may include red, orange, yellow, green, blue, indigo and violet. The present invention further includes a method of distinguishing oxygen tubing comprising the step of applying a spectrum of colors over the surface length of the tubing.

### BACKGROUND OF THE INVENTION

1. Field of Invention

The present invention relates to enhanced oxygen tubing for an oxygen concentrator.

2. Description of Related Art

Many individuals requiring medical care in their home or medical facility, i.e., hospital, an assisted living facility, or nursing home, may have to use oxygen therapy due to their particular medical needs. Many times the oxygen concentrators are used to provide a higher concentration of oxygen to the patient in a less expensive manner than using compressed air tanks. Tubing extends from the oxygen concentrator to supply the oxygen to the patient near their breathing area. A user of the oxygen concentrator many times maneuvers around and consequently the oxygen tubing extends from the concentrator over the floor or surface area near the patient. The traditional tubing used with an oxygen concentrator is transparent and therefore may not be as visible as based upon the background and coloring of the surface that it may lie upon. Due to the tubing's lack of visibility, individuals may trip over or accidentally step on the tubing that is being used by the patient. It would therefore be advantageous to have tubing available that is more visible than is presently used in the prior art.

Color tubing has been implemented in the medical field with respect to some particular applications. One example is disclosed in U.S. Pat. No. 6,668,832, which discloses an endotracheal tube, and a method for using the endotracheal tube where the tube includes a plurality of visually distinct regions that are closer to the proximal and the distal end. Another example is shown in U.S. Patent Application No. 20080097406 that discloses a color-coded medical apparatus for the administration of fluids where the tubing contains a visible marking by identification of fluid being administered from an intravenous bag through the tubing. It would be advantageous to apply the color application and color tubing techniques to a tubing use for oxygen concentrators in order to avoid tripping or injuries that might be associated with this transparent tubing.

### SUMMARY OF THE INVENTION

The present invention relates to oxygen tubing for an oxygen concentrator comprising: a spectrum of colors over the surface tubing, where the spectrum extends over the entire length of the tubing. The spectrum of colors may include red, orange, yellow, green, blue, indigo and violet. The present invention further includes a method of distinguishing oxygen tubing comprising the step of applying a spectrum of colors over the surface length of the tubing.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 depicts a portion of tubing according to the present invention.

### DETAILED DESCRIPTION

The present invention relates to multicolored tubing for use with oxygen concentrators. The colored tubing increases the visibility of the tubing and therefore helps to avoid tripping or stepping upon the tubing within a household. The colored tubing may feature a range of continuous colors in a spectrum sequence. Such an arrangement of color over the surface of the oxygen tubing increases the visibility of the tubing and reduces the likelihood of accidents.

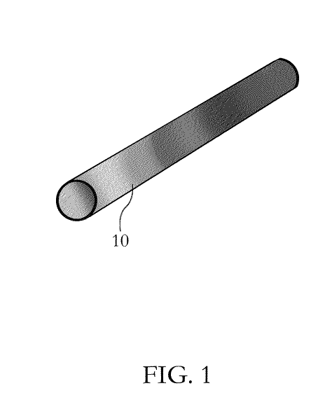

FIG. 1 depicts oxygen tubing according to the present invention. The oxygen tubing according to the present invention provides a spectrum color application over the surface thereof in order to increase the visibility of the tubing. The oxygen tubing 10 has a full sequence of colors ranging from red, orange, yellow, green, blue, indigo and violet. Because of this spectrum of colors, the tubing is readily more visible when used at a home or even at a health care facility. The colored tubing according to present invention may be manufactured of any available standard length ranging from 25 to 50 feet and the multicolored tubing stretches across the entire length of the oxygen tubing. Polyurethane may be used as the principle material to manufacture the tubing with the spectrum of rainbow colors within the tubing. The color spectrum makes the tubing easier to recognize against any background and therefore reduces or eliminates the likelihood of trips and falls due to lack of visibility of the oxygen tubing. The instant invention has been shown and described in what it considers to be the most practical and preferred embodiments. It is recognized, however, that departures may be made there from within the scope of the invention and that obvious modifications will occur to a person skilled in the art.

What is claimed is:

1. Oxygen tubing for an oxygen concentrator comprising:
a. a spectrum of colors over the surface tubing, where the spectrum extends over the entire length of the tubing.

2. The oxygen tubing according to claim 1, where said spectrum of colors include red, orange, yellow, green, blue, indigo and violet.

3. A method of distinguishing oxygen tubing comprising the step of:
a. applying a spectrum of colors over the surface length of the tubing.

4. The method according to claim 3, where said spectrum of colors include red, orange, yellow, green, blue, indigo and violet.
